(19) United States
(12) Plant Patent Application Publication
(10) Pub. No.: US 2016/0205833 P1
(43) Pub. Date: Jul. 14, 2016

(54) White delight one

(50) ;

(71) Applicant: The Texas A&M University System, College Station, TX (US)

(72) Inventors: David H. Byrne, Bryan, TX (US); Natalie Anderson, Calvert, TX (US)

(73) Assignee: The Texas A&M University System, College Station, TX (US)

(21) Appl. No.: 14/544,514

(22) Filed: Jan. 13, 2015

(51) Int. Cl. A01H 5/00

(52) U.S. Cl. PLT/195

(57) ABSTRACT

Disclosed is a new variety of Prunus persica named ‘WHITE DELIGHT ONE’. This new variety, which requires approximately 500-550 chilling units of dormancy, is considered to be a peach tree which produces a high quality, subacid, large, firm, clingstone, white-fleshed peach that matures in late May in the medium chill zone of Texas.

### BACKGROUND OF THE INVENTION

### Field of the Invention

This invention relates to peach trees and, more specifically, to peach trees referred to as a variety of Prunus persica named ‘WHITE DELIGHT ONE’ ‘WHITE DELIGHT ONE’, which requires approximately 500-550 chilling units of dormancy, produces an high quality, sub-acid, large, firm, clingstone, white-fleshed peach that matures in late May in the medium chill zone of Texas.

### SUMMARY OF THE INVENTION

The ‘WHITE DELIGHT ONE’ peach is characterized as to novelty and is otherwise noteworthy by producing fruit that ripens in the early season; is considered very high quality; and which is firm and has an attractive coloration. In this regard, the present variety of peach tree bears fruit that are ripe for commercial harvesting and shipment in the late May, when the fruit is grown in Fairfield, Tex. (medium chill zone of Texas). ‘WHITE DELIGHT ONE’ ripens 5 to 7 days earlier than ‘June Gold’ and 3 to 5 days after ‘Regal’ in the medium chill zone of Texas.

### ORIGIN OF THE VARIETY

The present peach tree was the result of an ongoing Stone Fruit Breeding Program of Texas A & M University, College Station, Brazos County, Tex. To this end, both controlled and hybrid crosses are made each year in order to produce seedling populations from which improved progenies are evaluated and selected.

‘White Delight One’ (WD1) tested as TX4C236LW is a 1998 cross between a yellow fleshed, medium chill peach selection (TX2492-1 =nectarine seedling ×Earligrande) and the Japanese cultivar ‘Chiyohime’ which is an early ripening subacid white-fleshed peach. ‘Chiyohime’ was released by the National Institute of Fruit Science (Tsukuba, Japan) in 1986 and is a hybrid between ‘Koyohakuto’ and ‘Saotome’ (Yoshida et al., 1987). Resulting seed from these crosses were planted in 1999 in a high density breeding orchard at the Texas A & M University Horticultural Farm in College Station, Tex. During 2003, this selection was marked as TX4C236LW for subsequent observation and noted as having exceptional characteristics. Two-year and older trees of the variety were subsequently evaluated during the 2005 through 2011 fruit growing seasons in both California (Fowler) and Texas (Fairfield, Terrell and College Station).

### ASEXUAL REPRODUCTION OF THE VARIETY

‘WHITE DELIGHT ONE’ was bud grafted onto virus-free Nemaguard (“The Brooks and Olmo Register of Fruit and Nut Varieties,” 3rd Ed., American Society of Horticultural Science Press, Alexandria, Va., 1997) peach rootstock in June 1998 at the nursery site in Oakdale, Calif. The variety was subsequently planted at the experimental orchard in the central portion of the San Joaquin Valley, near Fowler, Fresno County, Calif. and in three sites in Texas (College Station, Fairfield, and Terrell). Fruit from the resulting propagation has been evaluated during the period from 2005 to 2011 fruit seasons. This evaluation clearly demonstrated that the re-propagated trees were true to the characteristics of the original seedling in all observable aspects.

### BRIEF DESCRIPTION OF THE DRAWINGS

This new variety of peach tree is illustrated by the accompanying photographic drawings and depicts the plant by the best possible color representation using color photography, wherein:

FIG. 1. Color photograph of characteristic fruit with twig and leaves of ‘White Delight One’. The fruit shown shows sufficient external coloration for commercial harvest. The fruit are on a tree in Fowler, Calif. Note that the fruit are rounder than fruit produced in a lower chill zone in Fairfield, Tex. as shown in FIG. 2.

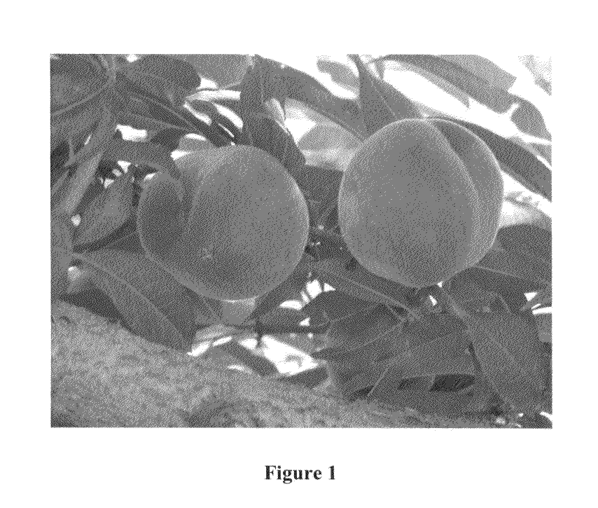

FIG. 2. Color photograph of ‘White Delight One’ fruit showing clingstone adhesion to flesh, internal flesh color, shape, and external coloring along suture. The fruit was harvested in the medium chill zone of Texas.

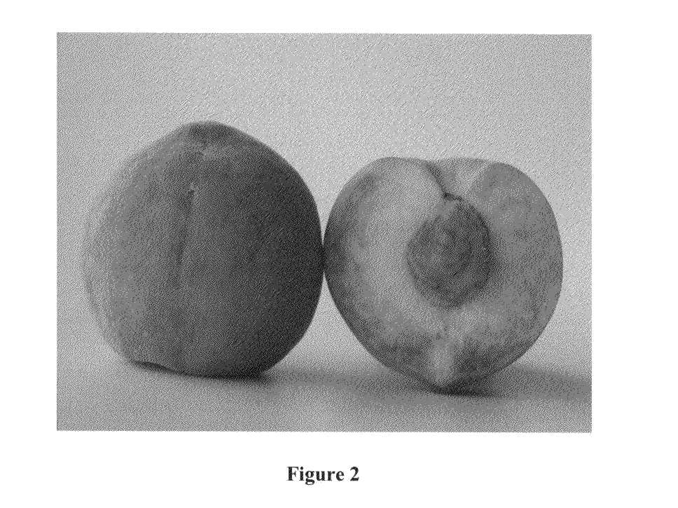

FIG. 3. Color photograph of the endocarps of ‘White Delight One’.

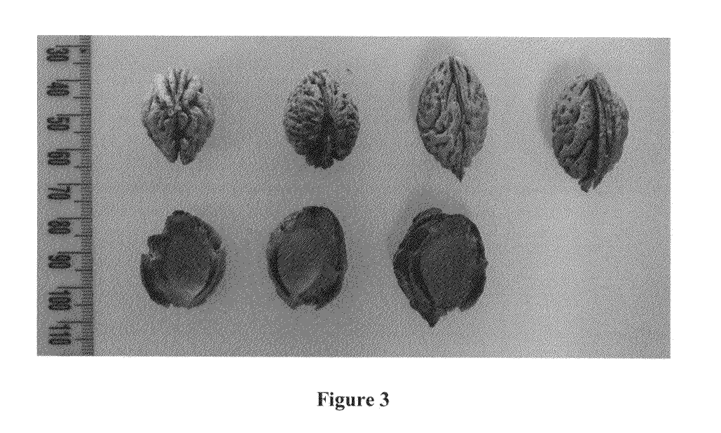

FIG. 4. A stem showing the leaves of the ‘White Delight One’ peach. The ruler is demarcated in millimeters.

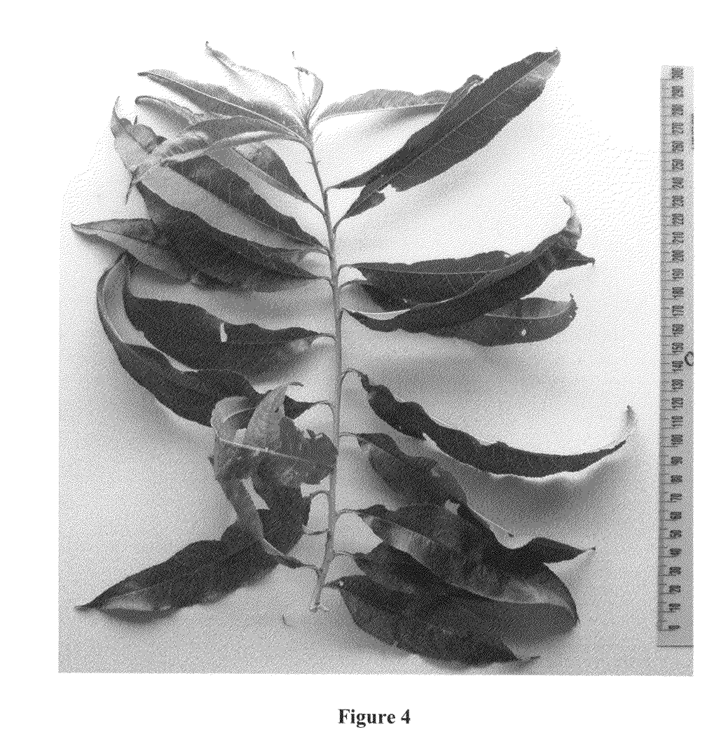

FIG. 5. Non-showy flowers of White Delight One’. The ruler is in millimeters.

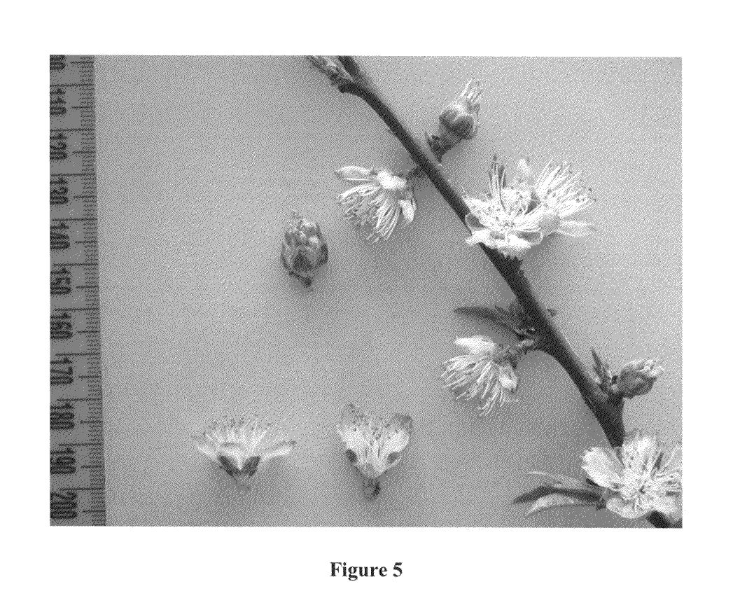

### BOTANICAL DESCRIPTION OF THE VARIETY

Referring more specifically to the pomological details of this new and distinct variety of peach tree, the following has been observed under the ecological conditions prevailing at the experimental orchards in the medium chill zone of Texas. All major color code designations are by reference to the R.H.S. Colour Chart (Third Edition) provided by The Royal Horticultural Society of Great Britain. Colors are approximate as color depends on horticultural practices such as light level and fertilization rate, among others.
Tree:
   Size.—Generally average to above average as compared to other common peach cultivars ripening in the early season of maturity.
   Height.—8 feet (2.44 m) at the end of the 2012 growing season.
   Width.—8 feet (2.44 m) at the end of the 2012 growing season.
   Vigor.—High.
   Density.—Medium to high.
   Productivity.—Productive.
   Shape.—The trees are vigorous with the typical semi-spreading growth habit similar to ‘TexKing’, ‘TexPrince’, and ‘TexRoyal’.
   Current season growth.—The current season growth for the new variety was approximately 4.25 to 4.6 feet (1.3-1.4 m).
   Regularity of bearing.—Regular, and considered hardy under typical conditions experienced in the medium chill zone of central Texas (College Station, Fairfield, Terrell) and in central San Joaquin Valley, Calif.
TRUNK:
   Size.—Approximately 4.9 inches (12.5 cm) in diameter and 18.0 inches (45.7 cm) in circumference when measured at a distance of approximately 12 inches (30.5 cm) above the soil level, at the end of the 2012 growing season on a five-year old tree.
   Bark texture.—Considered moderately rough with numerous folds of papery scarf-like skin being present.
   Bark coloration.—Variable, colors present are 166A of the Greyed-Orange Group, N186C, 187A and 187D of the Greyed-Purple Group, 198D of the Greyed-Green Group and N200D of the Brown group.
Branches:
   Size.—Considered medium for the variety.
   Thickness.—Average (about 6.5 cm in diameter as measured 10 cm from the trunk on a five-year old tree) as compared to other varieties.
   Surface texture.—Average and appearing furrowed on wood that is several years old.
   Lenticels.—Numerous flat, oval lenticels present. The lenticels range in size from approximately 3.0 to 9.0 mm in width and were approximately 1 mm in height.
   Current season shoots.—Surface texture—Substantially glabrous.
   Internode length.—Approximately 4.0 to 4.5 cm as measured in the middle of a current season stem.
   Color of mature branches.—The predominant colors are 165A and 177A-B of the Grey-Orange Group and 200D of the Brown group.
   Current season shoots.—Color—Light green (144A, N144D, 145A-B and 146C-D of the Yellow-Green Group) with some reddish-brown coloration appearing on exposed surface of the shoots (166C-D, 172B-C and 173A of the Greyed-Orange Group).
LEAVES:
   Size.—Considered moderately large for the species. Leaf measurements have been taken from vigorous upright current season growth approximately at mid-shoot.
   Leaf length.—Approximately 192 to 199 mm.
   Leaf width.—Approximately 52 to 60 mm.
   Leaf thickness.—Less than 1 mm.
   Leaf form.—Lanceolate.
   Leaf tip form.—Acuminate.
   Leaf upper surface color.—Medium green, approximately 137A-C of the Green Group.
   Leaf lower surface color.—Medium green varying among 137C-D and 138A-B of the Green Group.
   Leaf mid-vein color.—Light yellow green (145C-D, 149D and 150D of the Yellow-Green Groups).
   Leaf margins.—
   Form.—Considered crenate/crenulate.
   Uniformity.—Considered generally uniform.
   Leaf petioles.—
   Size.—Considered medium long.
   Length.—Approximately 12 mm.
   Thickness.—Approximately 2 mm.
   Color.—Pale green (145C-D, 149D and 150D of the Yellow-Green Groups).
   Leaf glands.—
   Size.—Approximately 1 mm in height and less than 1 mm in width.
   Number.—Generally 1-2 per leaf.
   Type.—Globose.
   Color.—Light brown (164A-B and 165B-C of the Greyed-Orange Groups).
   Leaf stipules.—
   Size.—Medium-long for the species.
   Length.—Approximately 12 to 14 mm.
   Form.—Lanceolate.
   Color.—Green (Yellow-Green Groups 144B-C and N144C) with reddish brown tips (Greyed-Orange Groups 164B-C and 165B) when young. The stipules are considered to be early deciduous.
   Ratio of wood (leaf) buds to flowering buds.—1 to 2 flower buds per vegetative bud.
Flowers:
   Floral buds.—
   General.—The floral buds are considered to be medium to medium large in size, conic in form, and slightly appressed relative to the bearing shoot.
   Color.—The bud scales are gray-brown, (approximately Brown Groups 200A and N200B). The buds are considered hardy under typical climatic conditions of the medium chill zone of Texas (College Station, Fairfield and Terrell) and the central San Joaquin Valley, Calif.
   Length.—Approximately 5 to 6 mm.
   Blooming type.—Considered medium early in relation to other peach cultivars commonly growing in the medium chill zone of Texas and the central San Joaquin Valley. Date of full bloom was generally late February to early March, or 5 to 7 days before ‘June Gold’ in the medium chill zone of Texas.
   Flower type.—Non-showy.
   Flower size.—Flower diameter at full bloom is approximately 20 to 25 mm.
   Bloom quantity.—Considered abundant.
   Flower bud frequency.—Normally 1 to 2 per node
   Petal size.—
   General.—Considered small to medium for the species.
   Width.—Approximately 6 mm.
   Length.—Approximately 11 to 12 mm.
   Petal form.—Broadly ovate.
   Petal count.—Nearly always 5.
   Petal color.—Medium pink (Red-Purple Group 62B-C, 65A-B and 68D).
   Petal claw.—
   Form.—The claw is considered truncate in shape and has a medium size when compared to other varieties.
   Length.—Approximately 1 to 1.5 mm.
   Width.—Approximately 1 mm.
   Petal margins.—Generally considered variable, from nearly smooth to slightly undulate.
   Petal apex.—Generally—The petal apices appear slightly domed.
   Flower pedicel.—
   Length.—Considered short, and having an average length of approximately 2 mm.
   Thickness.—Considered average, approximately 1 mm.
   Color.—Light green (Yellow-Green Group 144D and N144C-D).
   Floral nectaries.—
   Color.—Medium orange (Orange Group 25A-B, N25A-B and 28A-B).
   Calyx.—
   Surface texture.—Generally glabrous.
   Color.—A brownish red with green (Red Group 53A, Yellow-Green Group 144C, N144C-D, Greyed-Purple Group 183A-B, 184A-B and 185A).
   Sepals.—
   Surface texture.—The surface has a short, fine, wooly and a gray-colored texture.
   Size.—Average, and ovate in form.
   Color.—A brownish red with green (Red Group 53A, Yellow-Green Group 144C, N144C-D, Greyed-Purple Group 183A-B, 184A-B and 185A).
   Anthers.—
   General.—Average in size for the species.
   Color.—Golden yellow (Yellow Group 13A-B and Yellow-Orange Groups 14C and 16B-C).
   Pollen production.—Pollen is abundant, and is a yellow color (approximately Yellow Orange 16A).
   Filaments.—Size—Variable in length, approximately 11 to 13 mm, with the filaments slightly shorter than or equal to the pistil.
   Color.—White (approximately White Group 155A-D) and becoming dark pink (Red-Purple Group 70B and 72C-D) with advanced maturity.
   Pistil.—
   General.—Average in size, but slightly longer or equal to the anther height.
   Length.—Approximately 15 to 18 mm, including the ovary.
   Color.—Considered a very light green (Yellow-Green Group 151A).
   Surface texture.—The variety has a long, silver white pubescent pistil (approximately White Group 155A-D).
Fruit:
   Maturity when described.—The present variety of fruit is described, as it would be found in its firm ripe condition at full commercial maturity. Under the ecological conditions prevailing in the medium chill of Texas, this cultivar is picked from mid-May until early June depending on the year and the site of the orchard. The average time of harvest for Fairfield, Tex. was May 25th which was 3-5 days after ‘Regal’ and 5-7 days before ‘June Gold’.
   Size.—General—Medium large to large for the season and considered uniform.
   Average cheek diameter.—Approximately 56 to 59 mm.
   Average suture diameter.—Approximately 57 to 62 mm.
   Average axial diameter.—Approximately 51 to 53 mm.
   Fruit form.—Generally considered round-oblate with unequal halves. Occasionally the fruit exhibits less symmetry when comparing the suture height with the line opposite the suture. The fruit is generally uniform in symmetry when viewed from the apical aspect.
   Fruit suture.—Generally, the suture appears as a thin line that extends from the base to the apex, and appears deeper at the apex, forming a shallow basin at the apical point. No apparent callusing or stitching exists along the suture line.
   Color.—Generally the ground and blush colors.
   Ventral surface.—Form—Considered uniform.
   Stem Cavity.—Size—Considered shallow for the species.
   Width.—Approximately 8 to 9 mm.
   Length.—Approximately 14 to 16 mm.
   Depth.—Approximately 6 to 11 mm.
   Fruit base.—Round and flat.
   Fruit apex.—Flat.
   Fruit stem.—Length—4 to 5 mm Thickness—Approximately 3 mm.
   Fruit skin.—Generally considered medium or average in thickness. Surface Texture — Heavy, medium pubescence. Skin Acidity — Considered neutral. Tenacious to Flesh — Yes at commercial maturity. Tendency to Crack — Not observed. Skin Color — Generally — Variable, with approximately 40-90% of the fruit surface covered with an attractive orange red blush. Down — Light and short Blush Color — The blush color ranges from dark red to medium red (Orange-Red Group N34A and Red Groups 39A-B, 46A-B and 47A) with many degrees of shading and blending between these colorations. Skin Ground Color — The skin ground color is a medium yellow to yellow green color (Green-Yellow Group 1C-D and Yellow Groups 2C-D and 3C-D).
   Flesh color.—Considered white to ivory in color (White Group 155A-B and Yellow-White Group 158B-D) with red flecks appearing throughout becoming more abundant near the skin (Similar to Red-Purple Groups 58A and 63A).
   Flesh fibers.—Present, numerous and lightly colored. These fibers are present throughout the flesh.
   Stone cavity color.—Generally a yellowish green to medium yellow (Green-Yellow Group 1C-D and Yellow Groups 2C-D and 3C-D) and darkening upon exposure to air.
   Flesh texture.—Generally, the flesh is considered from and fine at commercial maturity.
   Ripening.—Generally the fruit of the present variety ripens evenly.
   Flavor.—Considered very sweet and with subacid flavor.
   Aroma.—Pleasant and reasonably abundant.
   Eating.—Generally considered very good, particularly for an early ripening variety.
Stone:
   Attachment.—Clingstone at commercial maturity.
   Stone size.—Generally considered medium-large relative to the ratio of stone to fruit size.
   Length.—Approximately 34 to 36 mm.
   Width.—Approximately 23 to 27 mm.
   Thickness.—Approximately 17 to 19 mm.
   Fibers.—Generally a few medium length fibers are sporadically attached along the surface of the stone.
   Stone form.—Oblate-elliptical.
   Stone base.—The stone is medium to wide.
   Apex shape.—Narrow to medium.
   Stone shape.—The stone normally elongated.
   Stone surface.—
   Surface texture.—Single pits and rosettes of pits.
   Ridges.—Numerous fine ridges are present basally, and converge towards the base of the stone.
   Ventral edge.—Small to medium.
   Dorsal edge.—Shape—Grooved and having moderately rough edges.
   Stone color.—The color of the dry stone is a medium brown color (Greyed-Orange Groups 164B and 165B-C). The color of the inside surface of the endocarp is primarily Greyed-Orange 165B-C.
   Tendency to split.—Splitting is relatively uncommon.
   Kernel.—the kernel fills the endocarp at harvest and measures approximately 4-5 mm in thickness, 11-13 mm in width, and 15-16 mm in length. When dried the shriveled kernels measure approximately 1 mm in thickness, 10-11 mm in width, and 14-16 mm in length. The colors of the shriveled kernels are primarily 164A and 165B-C of the Greyed-Orange Group and N199C-D of the Grey-Brown Group.
   Use.—The subject variety, ‘WHITE DELIGHT ONE’, is considered to be a peach tree of early-season maturity, which produces subacid white-fleshed fruit which are firm, attractively colored suitable for the fresh fruit market.
   Keeping quality.—Good.
   Resistance to insects and disease.—No particular susceptibilities were noted.
   Shipping quality.—Average.

Although the new variety of peach tree possesses the described characteristics when grown under the ecological conditions prevailing in the mediun chill zone of Texas, it will be understood that variations of the usual magnitude and characteristics incident to the changes in growing conditions, fertilization, pruning, and pest control are to be expected.

### REFERENCES

Anon. 1995. R.H.S. Colour Chart. The Royal Hort. Soc., London.

Yoshida, M., M. Yamaguchi, H. Kyotani, T. Kozono, T. Nishida, and Y. Ishizawa. 1987. New peach cultivar ‘Chiyohime’. Bull. Fruit Tree Res. Sta. Series A, 14:1-8.

We claim:

1. A new and distinct Prunus persica tree, substantially as illustrated and described herein.
